(19) United States
(12) Patent Application Publication
(10) Pub. No.: US 2014/0378901 A1
(43) Pub. Date: Dec. 25, 2014

(54) MECHANICAL PUMP TO TUBE INTERFACES, SYSTEMS INCLUDING THE INTERFACES AND METHODS FOR PRODUCING SAME

(71) Applicant: Q-CORE MEDICAL LTD., Netanya (IL)

(72) Inventors: Shachar Rotem, M.P. Hefer (IL); Omer Havron, Tel Aviv (IL)

(73) Assignee: Q-CORE MEDICAL LTD., Netanya (IL)

(21) Appl. No.: 13/924,572

(22) Filed: Jun. 23, 2013

(51) Int. Cl. A61M 5/142; A61M 39/12; A61M 39/10

(52) U.S. Cl. CPC A61M 5/14228; A61M 39/1011; A61M 39/12
USPC 604/151; 604/533

(57) ABSTRACT

The present invention includes systems, devices and methods for mechanically interfacing between a pump and an infusion kit or conduit, and methods and materials for producing same. According to some embodiments of the present invention, there may be provided a mechanical pump-tube interface unit adapted to detachably connect to a pump. The mechanical pump-tube interface unit may be adapted to hold a fluid conduit/tube positioned such that when the interface is attached to a pump the pumping mechanism of the pump acts upon the fluid in the tube. A mechanical pump-tube interface unit may include physical adaptations designed to prevent longitudinal and/or rotational movement, in relation to the unit, of a fluid conduit/tube held by or attached to the interface unit. According to further embodiments, a segment of conduit/tube may be attached/connected to a mechanical tube-interface during the assembly of the interface unit, to form one integral unit including the conduit/tube segment secured/trapped within.

### FIELD OF THE INVENTION

The present invention generally relates to the field of medical devices. More specifically, the present invention relates to mechanical pump to tube interfaces, systems including the interfaces and methods for producing same.

### BACKGROUND

Various types of medical infusion pumps are known in the art. One common type of infusion pump is a peristaltic pump, in which fluid is made to flow through an elastic tube by external compression of the tube. Typically, a peristaltic mechanism, such as a set of cams or fingers, compresses the tube in a cyclic pattern at a sequence of locations along the length of the tube, so as to cause the fluid to flow through the tube at a desired volumetric rate.

When incorporated into medical systems, peristaltic pumps are often used to pump liquids within an infusion system. The relative accuracy and controllability of such pumps makes them desirable for such implementations, as the administration of pharmaceutical liquids to patients requires accuracy and strict regulation of flow. In such implementations, the pumping mechanism is usually reusable, whereas the infusion tubes are typically disposable. Accordingly, it is necessary to interface between the infusion tubes and the pump. It is therefore desirable to provide pump to infusion tube interfaces which promote accuracy and reliability of the system which are yet easy to use.

### SUMMARY OF THE INVENTION

One advantage of peristaltic pumps in medical applications is that the pump mechanism is typically external to the fluid conduit (e.g. flexible tube), thus preserving the sterility of the fluid flowing through the conduit/tube. The conduit is typically part of a disposable infusion kit, while the pump itself (which may include the complete pumping mechanism, as well as a pressure sensor module and/or an air bubble sensor) may be reused many times. Embodiments of the present invention that are described hereinbelow provide systems, devices and methods for attaching/interfacing an infusion tube to a pump, in such a manner that the tube is placed precisely in the right location in relation to the pump, every time. Further, it may be desirable to maintain a precise location of the tube in relation to the pump, throughout its use, to promote accuracy and stability of the system. Further, as many different caretakers may be using the system, having different levels of training and competence, it may be desirable to limit as much as possible the possibility of misplacing or improperly aligning the tube. Currently, such interfaces are generally lacking in these goals. It is therefore desirable to provide a more accurate and foolproof pump to tube interface for peristaltic pumps.

The present invention includes systems, devices and methods for mechanically interfacing between a pump (e.g. a peristaltic pump) and an infusion kit or conduit, and methods and materials for producing same. According to some embodiments of the present invention, there may be provided a mechanical pump-tube interface unit adapted to detachably connect to a pump. The mechanical pump-tube interface unit may be adapted to hold a fluid conduit/tube positioned such that when the interface is attached to a pump the pumping mechanism of the pump acts upon the fluid in the tube. For example, the interface may hold the conduit/tube atop a series of “fingers” of a peristaltic pump such that a cyclical motion of the fingers causes fluid to flow through the conduit/tube. A mechanical pump-tube interface unit may include physical adaptations designed to prevent longitudinal and/or rotational movement, in relation to the unit, of a fluid conduit/tube held by or attached to the interface unit. According to further embodiments, a segment of conduit/tube may be attached/connected to a mechanical tube-interface during the assembly of the interface unit, to form one integral unit including the conduit/tube segment secured/trapped within.

According to some embodiments, a mechanical pump-tube interface unit may include mechanical elements/adaptations to connect and secure the interface unit to a pump. For example, a mechanical pump-tube interface unit may include latches and connectors matching hinges, latches and connectors upon a pump. Such mechanical elements and adaptations may be configured to connect the mechanical pump-tube interface unit to the pump such that when the interface unit is attached to the matching pump the pumping mechanism of the pump is positioned to act upon the fluid in the tube. For example, a hinge type receptacle may be fixed to the pump body on one side of the peristaltic mechanism, defining a hinge axis, and a catch may be fixed to the pump body on the far side of the peristaltic mechanism. According to some embodiments, a mechanical pump-tube interface unit may be configured to hold or include a portion of conduit/tube, and include a hinge tongue, which is configured to engage the hinge type receptacle together creating a hinge between the pump and interface unit. A catch receptacle, also included in the mechanical pump-tube interface unit may be configured to receive and secure the catch upon rotation of the mechanical interface unit about the hinge axis while the hinge tongue is engaged to the hinge type receptacle, so as to bring the conduit/tube into engagement with the peristaltic mechanism.

In some cases, the peristaltic mechanism may include multiple fingers, which are driven to compress and release the conduit/tube in a predetermined cyclic pattern.

In some embodiments, a mechanical pump-tube interface unit may be shaped to match a shape of the pump mechanism it is intended to interface to. For example, when the peristaltic mechanism has a linear configuration, the mechanical interface may have an elongated shape corresponding to the linear configuration of the peristaltic mechanism.

In some embodiments, the pump body may include a rim surrounding the peristaltic mechanism, and an interface segment of conduit (defined below) may include flanges or may include connectors having flanges, fixed to opposing ends of a portion of the tube within the mechanical pump-tube interface unit which flanges may lodge against the rim when the unit is attached to the pump. The infusion pump may further include a door or cover, which may close over the rim so as to enclose the peristaltic mechanism. Such a rim may have openings shaped to receive a conduit/tube so that a connected conduit/tube extends through the openings when the door is closed. According to some embodiments, the flanges may be configured to lodge inside or outside the rim and have respective diameters that are larger than the openings so as to prevent longitudinal motion of the conduit/tube after the door has been closed.

According to some embodiments, a mechanical pump-tube interface unit may include an anti-free-flow mechanism, which may be configured to, when engaged, prevent flow of the fluid through the portion of the tube secured within the interface unit. The anti-free-flow mechanism may be adapted to automatically engage any time the interface unit is disconnected from a pump and automatically disengage whenever the unit is connected to a pump. According to some embodiments, the anti-free-flow mechanism may have an override mechanism to allow disengagement of the anti-free-flow mechanism without connecting the interface to the pump, i.e. while the interface unit is not connected. According to further embodiments, the mechanical pump-tube interface unit may further include an anti-free-flow override locking mechanism designed to lock the override mechanism of the anti-free-flow mechanism in an overriding position. According to yet further embodiments, such a lock may automatically unlock when the mechanical pump-tube interface unit is connected to a pump. Accordingly, a user may actuate and lock the override mechanism while the interface unit is disengaged from a pump. In this position the fluid may be free to flow through the conduit/tube without any further action from the user, however, according to these embodiments, if the mechanical pump-tube interface unit is connected to a pump, the override mechanism may automatically unlock such that if the interface unit is again disconnected from the pump, the anti-free-flow mechanism will engage normally and block fluid flow in the conduit/tube unless the override mechanism is actuated by the user once again.

According to some embodiments, there may be provided a section of conduit/tube designed to reside within a mechanical pump-tube interface unit (hereinafter referred to as an “interface segment”). According to further embodiments, an interface segment of a conduit/tube may be comprised of a material suitable for interacting with a pump, such as Silicone materials. It should be understood that the rest of the conduit/tube (i.e. a portion intended to reside outside the pump-tube interface unit) may be comprised of a different material (e.g. pvc).

a portion of conduit/tube intended to conduct fluid from a reservoir to the mechanical pump-tube interface unit (i.e. pump) will hereinafter be referred to as a “supply line”.

a portion of conduit/tube intended to conduct fluid from the mechanical pump-tube interface unit (i.e. pump) to a patient will hereinafter be referred to as a “patient line”.

According to some embodiments, an interface segment of conduit/tube may include or be connected, at one or both of its ends, to connectors adapted to connect the interface segment of conduit/tube to a supply line on one side and a patient line on the other. Such connectors may include flanges which may function as described above in relation to flanges, i.e. may lodge inside or outside a rim of the pump and have respective diameters that are larger than the openings so as to prevent longitudinal motion of the conduit/tube.

According to some embodiments, a mechanical pump-tube interface unit may include mechanical adaptations (Traps) adapted to secure/trap an interface segment of conduit/tube within the interface unit and prevent and/or inhibit longitudinal and/or rotational movement of an interface segment of conduit/tube residing within the interface unit. Such mechanical adaptations (traps) may be designed to act on the tube itself and/or upon one or both of the connectors at its ends. For example, a mechanical pump-tube interface unit may include grooves (e.g. a u shaped groove) on one or both sides designed to receive the end connectors/flanges and secure/trap them in place, thus preventing longitudinal movement of the interface segment of conduit/tube within the unit and possibly further preventing or inhibiting rotational movement of the interface segment of conduit/tube in relation to the interface unit.

According to further embodiments, a method of assembling a mechanical pump-tube interface unit may be provided. According to some embodiments, an interface segment of conduit/tube, including connectors, may be attached to a mechanical pump-tube interface unit, sterilized and packaged together. The interface segment of conduit/tube may be secured/trapped within the mechanical pump-tube interface unit in its final position by appropriate mechanical fasteners, grooves and/or any other suitable mechanical configuration. Subsequently, when ready to be used, the mechanical pump-tube interface unit may first be attached to a supply and patient line by means of the connectors on the ends of the interface segment of conduit/tube and then connected to a pump as described above. The interface segment of conduit/tube may thus become an integral part of the mechanical pump-tube interface unit.

There is additionally provided, in accordance with some embodiments of the present invention, a method for infusion, including providing a mechanical pump-tube interface unit, which holds a portion of a flexible infusion tube and includes a hinge tongue and a catch receptacle. The hinge tongue may be inserted into a hinge type receptacle, which defines a hinge axis, on an infusion pump. The mechanical pump-tube interface unit may be rotated about the hinge axis while the hinge tongue engages the hinge type receptacle until the catch latches upon the catch receptacle, so as to bring the tube into engagement with a peristaltic mechanism of the infusion pump. The infusion pump is actuated while the tube is in engagement with the peristaltic mechanism so as to propel a fluid through the tube.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic illustration of an exemplary medical infusion system connected to a patient, including a mechanical pump-tube interface unit, all in accordance with some embodiments of the present invention;

FIGS. 2A-2H are illustrations of an exemplary mechanical pump-tube interface unit, from different angles, with and without a conduit/tube attached thereto, all in accordance with some embodiments of the present invention;

FIGS. 3A-3B are illustrations of cross sections of an exemplary mechanical pump-tube interface unit, with and without a conduit/tube attached thereto, all in accordance with some embodiments of the present invention;

FIGS. 4A-4B are enlarged illustrations of a conduit/tube trap section of an exemplary mechanical pump-tube interface unit, with and without a conduit/tube attached thereto, all in accordance with some embodiments of the present invention;

FIG. 5 includes illustrations of components of an exemplary mechanical pump-tube interface unit, prior to assembly, all in accordance with some embodiments of the present invention;

FIGS. 6A-6K are illustrations of an exemplary assembly process of an exemplary interface segment of conduit and its assembly to a mechanical pump-tube interface unit, all in accordance with some embodiments of the present invention;

FIGS. 7A-7B are flowcharts including exemplary steps of assembly of a mechanical pump-tube interface unit, all in accordance with some embodiments of the present invention;

FIG. 8 is an illustration of an exemplary peristaltic pump and an exemplary mechanical pump-tube interface unit positioned and aligned to be connected to the pump in the appropriate location, all in accordance with some embodiments of the present invention;

FIG. 9 is an illustration of an exemplary peristaltic pump and an exemplary mechanical pump-tube interface unit connected to the pump in the appropriate location, all in accordance with some embodiments of the present invention;

FIGS. 10A-10C are illustrations of an exemplary front loading embodiment of an exemplary mechanical pump-tube interface unit, wherein FIG. 10A presents the assembled unit, FIG. 10B is an enlargement of the trap section of the exemplary mechanical pump-tube interface unit presented in FIG. 10A and FIG. 10C presents the connectors and conduit/tube segments separately from the rest of the unit and also includes an enlarged drawing of the exemplary connector, all in accordance with some embodiments of the present invention;

FIGS. 11A-11H are enlarged illustrations of different embodiments of connectors and matching conduit/tube trap sections of exemplary mechanical pump-tube interface units, all in accordance with some embodiments of the present invention;

FIGS. 12-12A include an enlarged illustration of a connector and matching conduit/tube trap section of an exemplary mechanical pump-tube interface unit and a set of cross-section illustrations of the illustrated trap section, demonstrating different configurations of the connector and matching trap, all in accordance with some embodiments of the present invention;

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

It should be understood that the accompanying drawings are presented solely to elucidate the following detailed description, are therefore, exemplary in nature and do not include all the possible permutations of the present invention.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, components and circuits have not been described in detail so as not to obscure the present invention.

In the following detailed description references to the figures appear in brackets. Numbers or letters appearing in brackets, e.g. [500], excluding paragraph numbers, should be understood to refer to elements marked within the figures by the same number or letter which appears in the brackets.

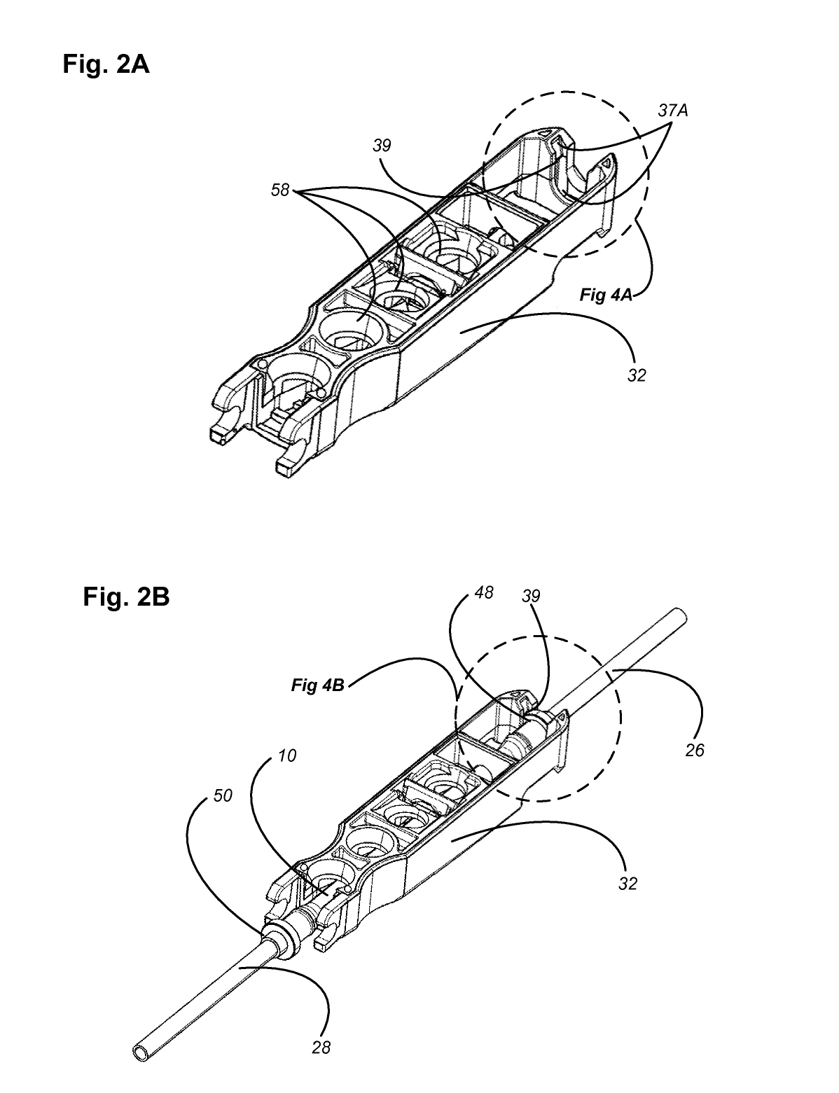

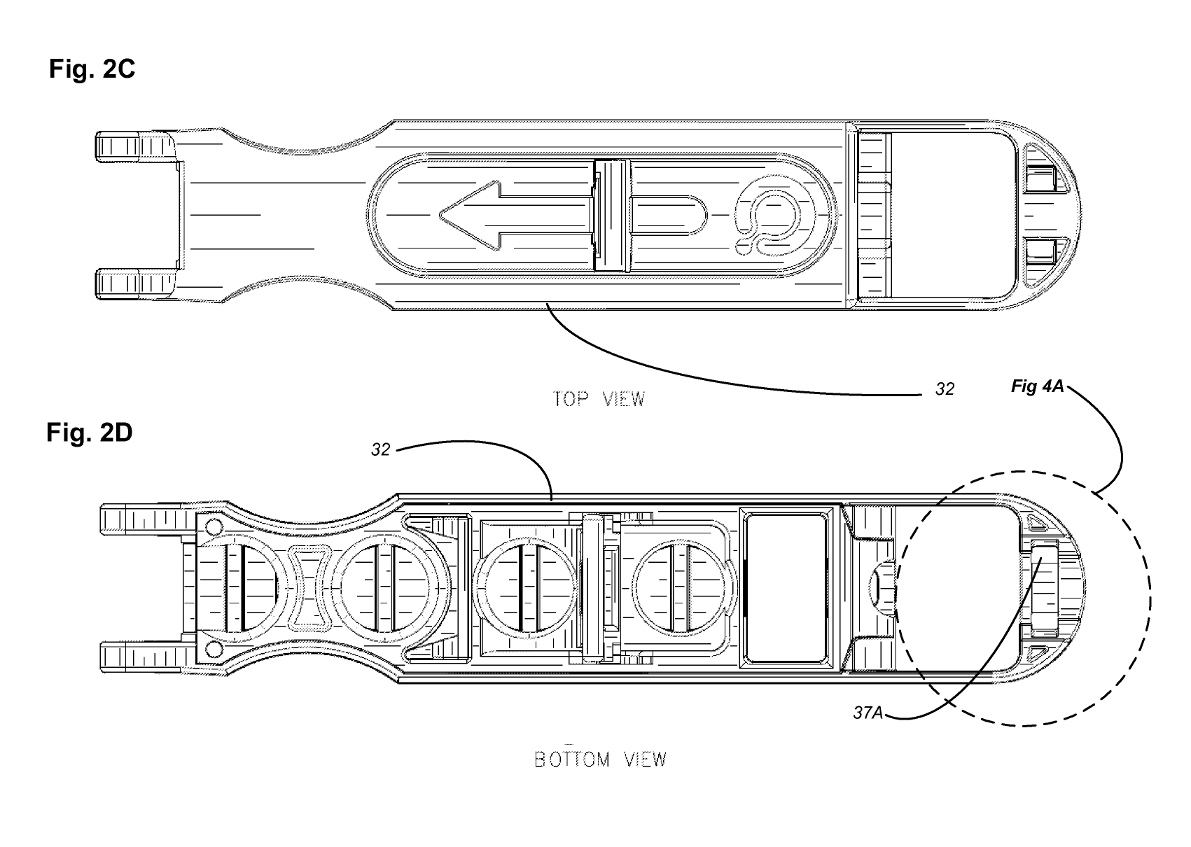

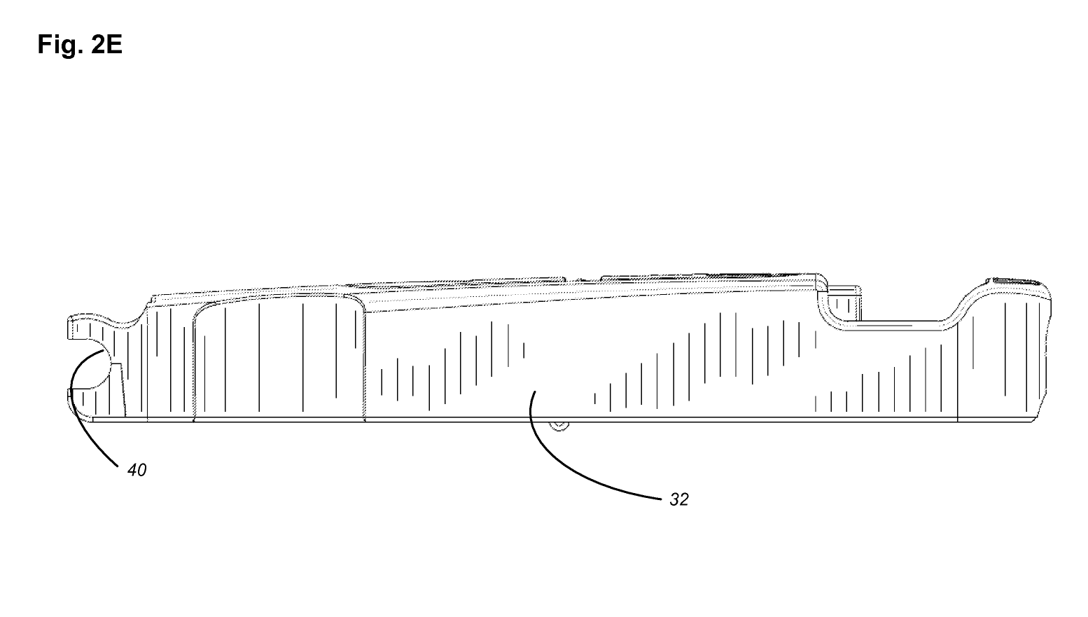

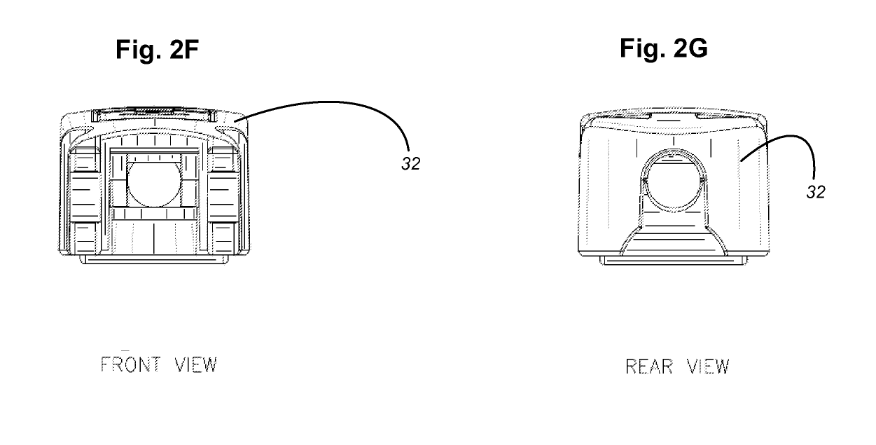

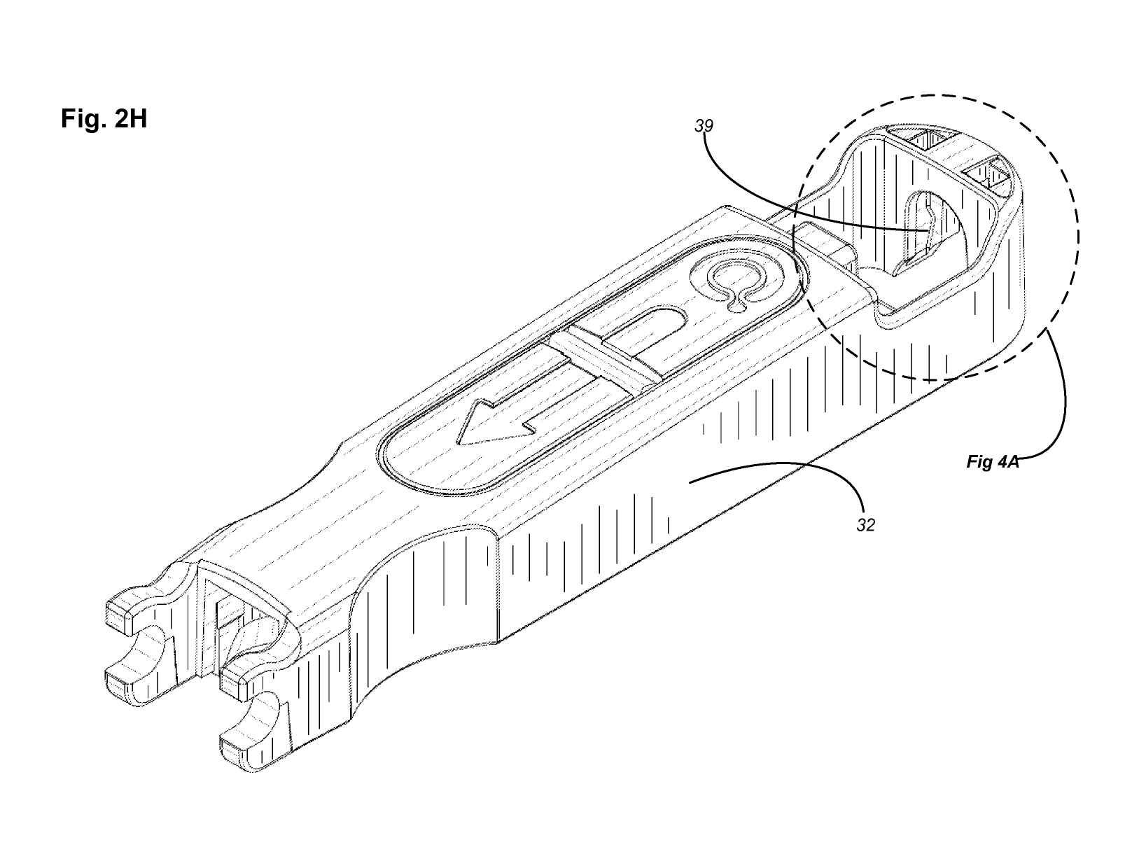

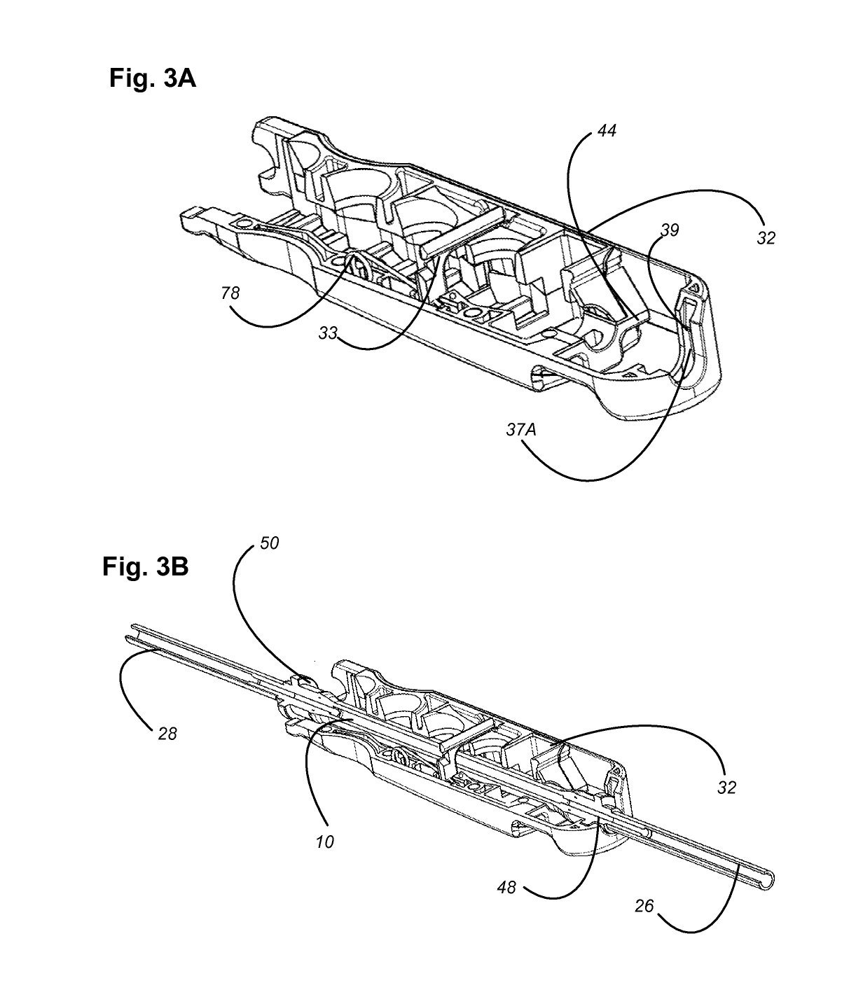

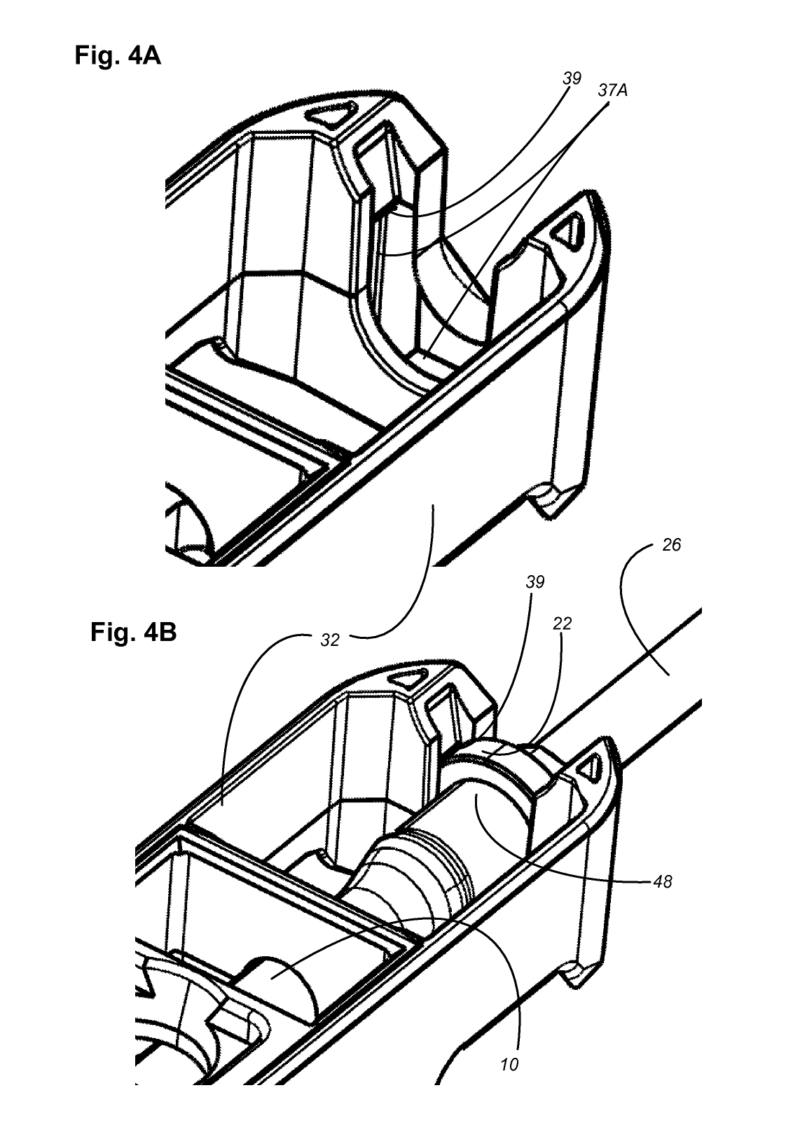

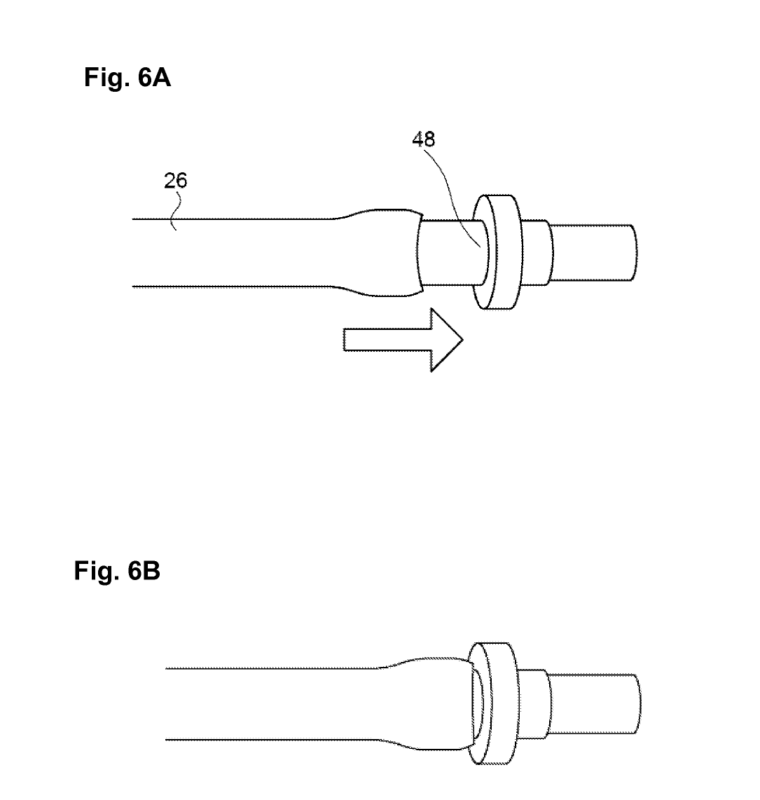

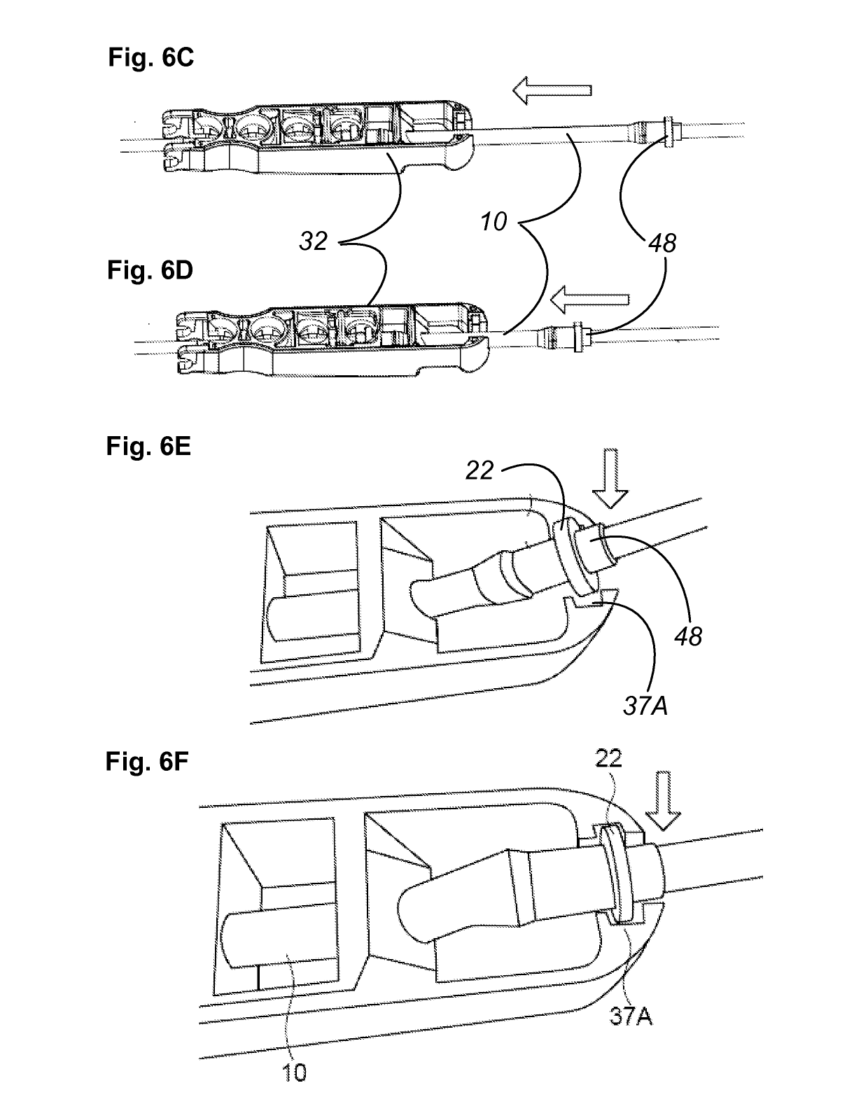

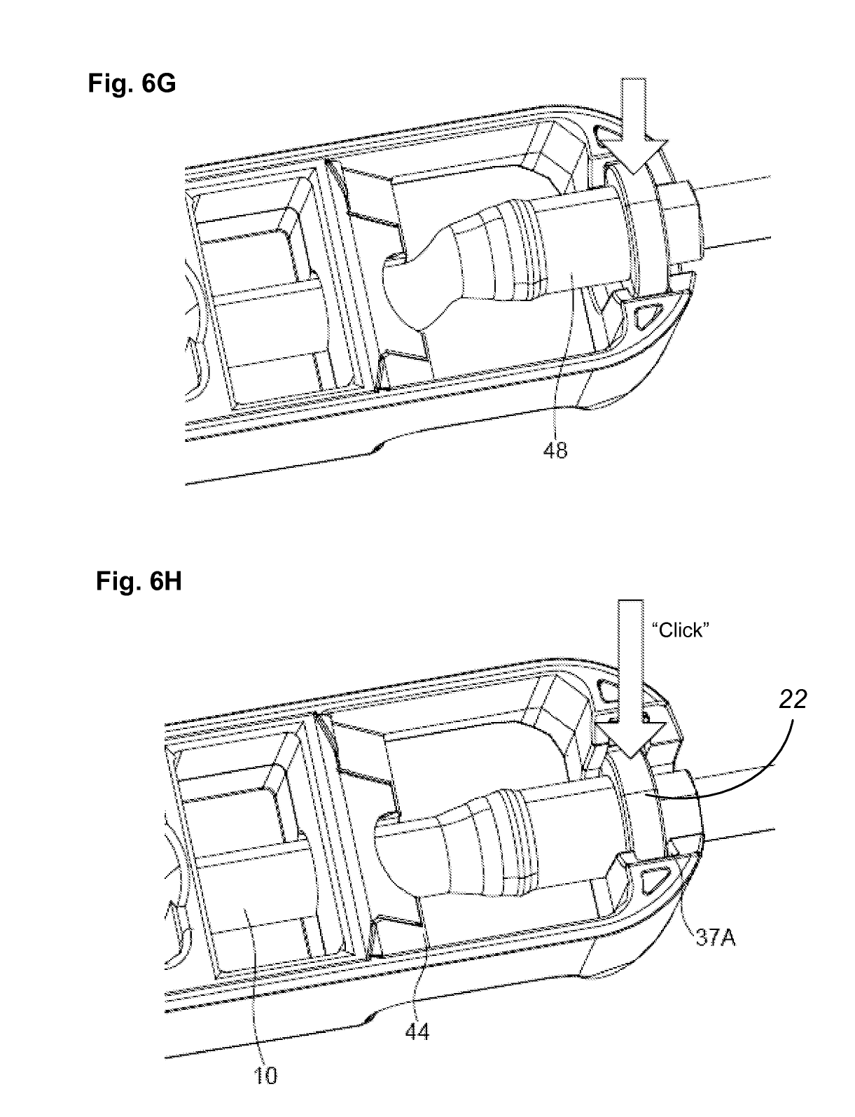

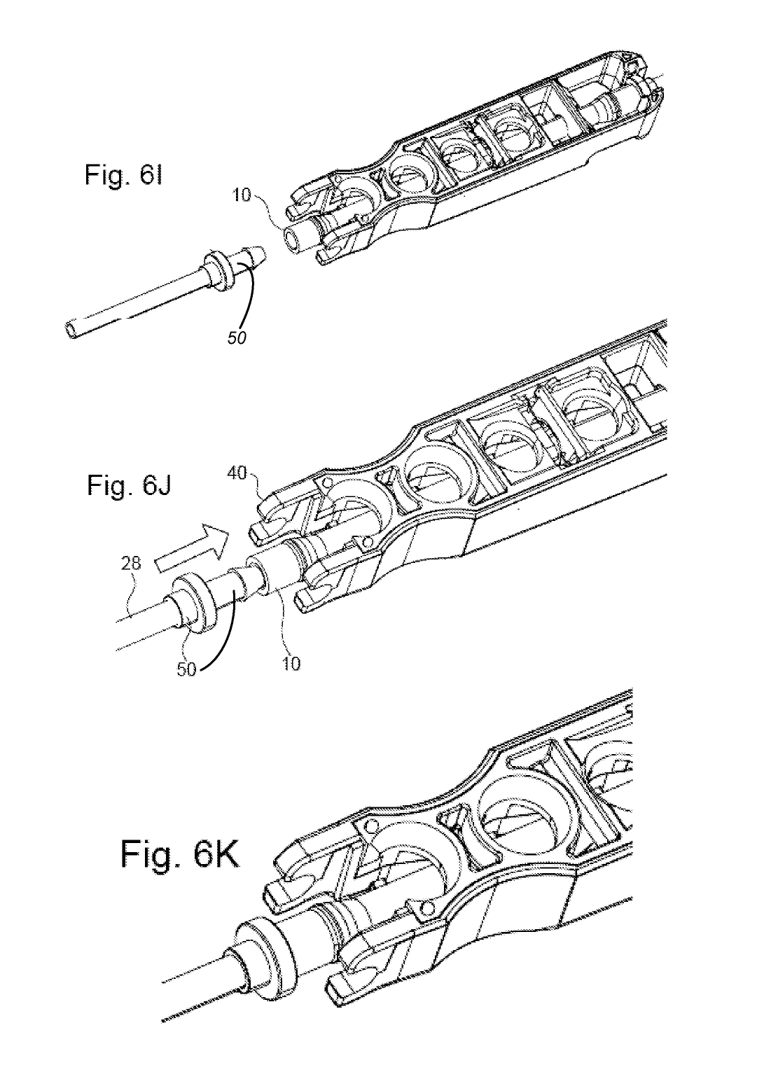

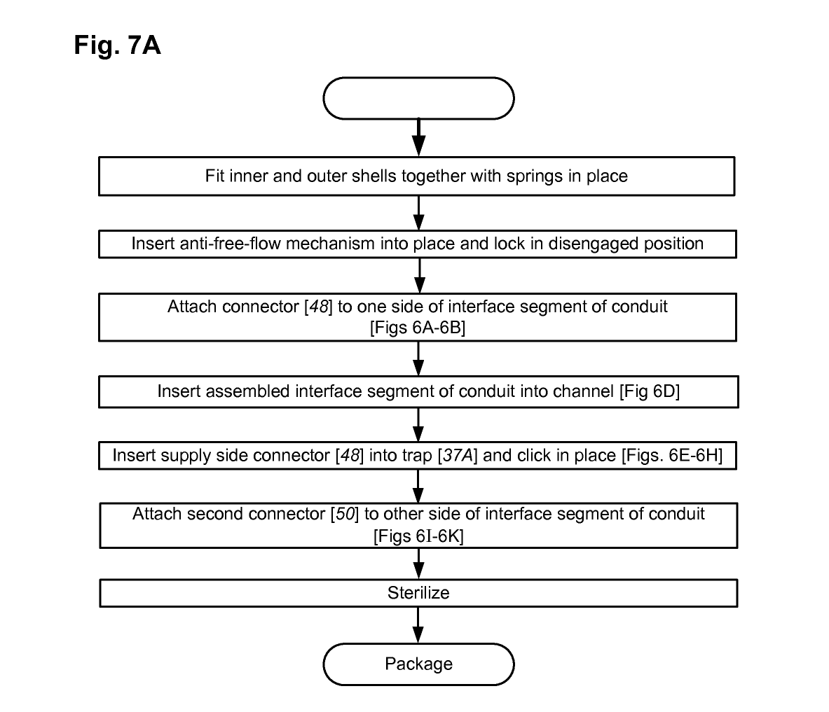

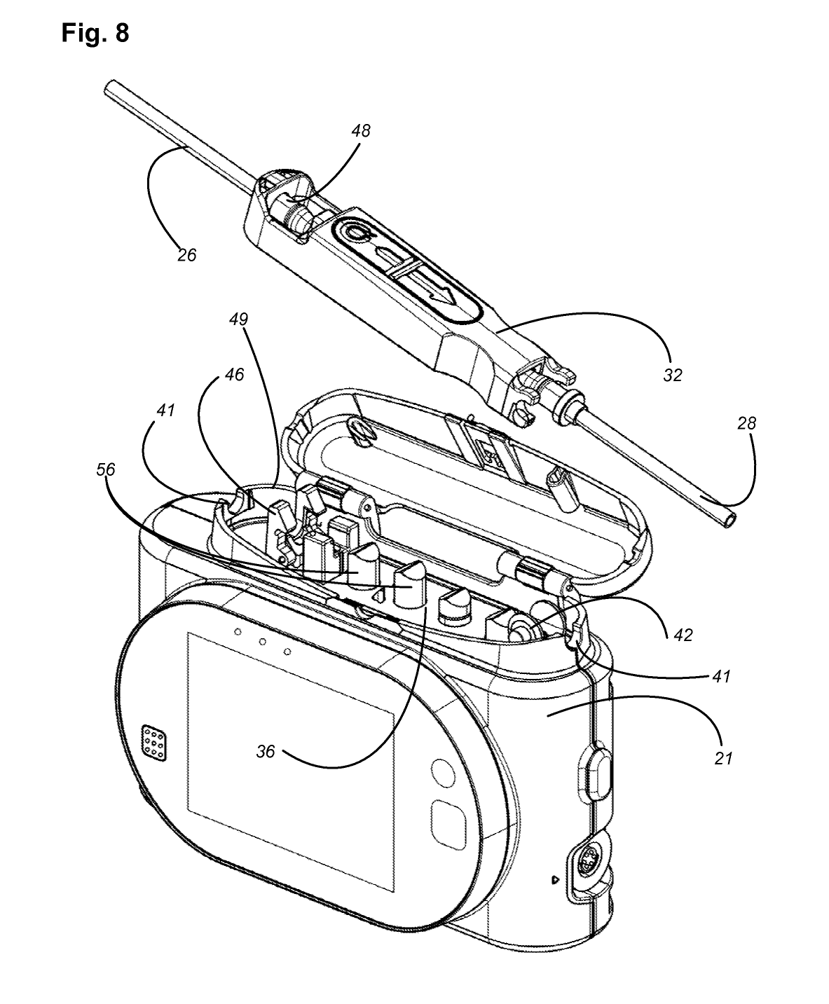

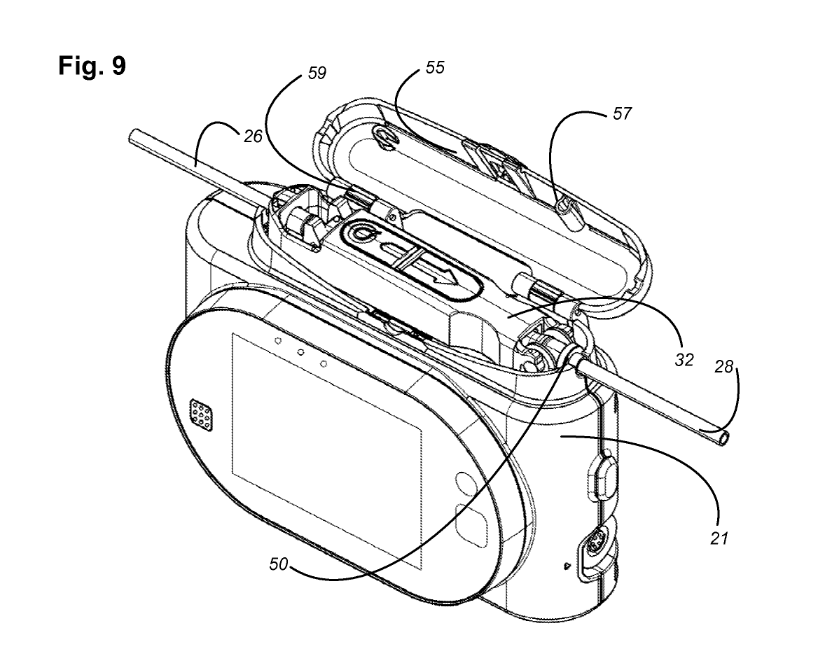

The present invention includes systems, devices and methods for mechanically interfacing between a pump (e.g. a peristaltic pump) and a medical infusion kit or conduit, and methods and materials for producing same. According to some embodiments of the present invention, there may be provided a mechanical pump-tube interface unit [see FIGS. 2A-2H for example] adapted to detachably connect to a pump [see FIGS. 8-9 for example]. The mechanical pump-tube interface unit may be adapted to hold a fluid conduit/tube positioned such that when the interface is attached to a pump, the pumping mechanism of the pump acts upon the fluid in the tube causing it to flow through conduit/tube. For example, the interface may hold the conduit/tube atop a series of “fingers” of a peristaltic pump such that a cyclical motion of the fingers causes fluid to flow through the conduit/tube [see FIG. 9 for example]. A mechanical pump-tube interface unit may include physical adaptations designed to secure/trap a fluid conduit held within the unit and thus prevent and/or inhibit longitudinal and/or rotational movement of the fluid conduit/tube [see FIGS. 4A-4B for example]. According to further embodiments, a segment of conduit/tube may be attached/connected to a mechanical pump-tube interface unit during the assembly of the interface unit, to form one integral unit including the conduit/tube segment secured within [see FIGS. 6A-6K for example].

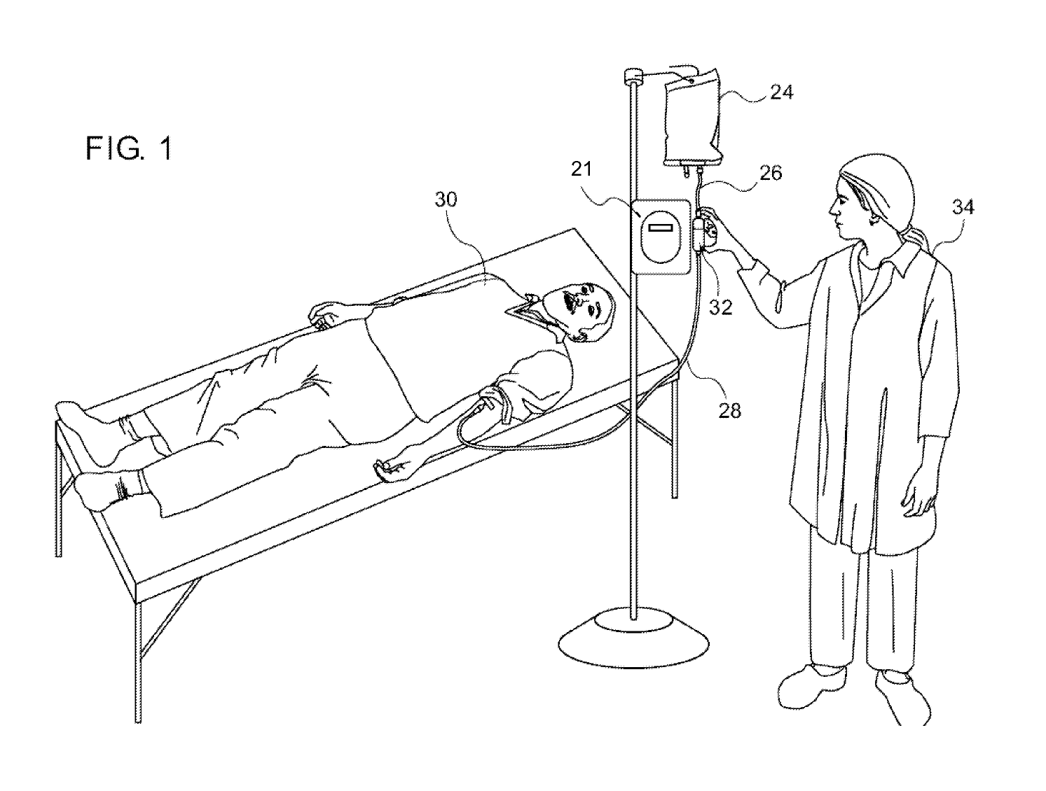

FIG. 1 is a pictorial illustration of a medical infusion system [20], in accordance with some embodiments of the present invention. System [20] may comprise a peristaltic infusion pump [21], which may pump an infusion fluid from a reservoir [24], through an upstream tube segment [26] (commonly referred to as the “supply line”) and a downstream tube segment [28] (commonly referred to as the “patient line”), into a vein of a patient [30]. This particular type of infusion system is shown here by way of illustration, but the principles of the present invention, as described hereinbelow, may likewise be applied to other types of peristaltic pumps and in substantially any sort of application that uses such pumps, such as delivery of drugs and of both enteral and parenteral nutrition. Although the pictured embodiment represents a clinical environment, the devices and methods described herein are also suitable for ambulatory and home use, particularly since they can operate even when the pump and reservoir are at the same level as or lower than the patient.

Tube segments [26] and [28] may be connected to a mechanical pump-tube interface unit [32] or to an interface segment of conduit/tube residing within the interface unit. The mechanical pump-tube interface unit may couple to pump [32] in a manner that is shown and explained below in greater detail. Unit [32] may contain an interface segment of conduit/tube (not shown in FIG. 1) that may be connected in series with tube segments [26] and [28], thus creating a flow path from reservoir [24] to patient [30]. In some embodiments, tube segments [26] and [28] may be fabricated from polyvinyl chloride (PVC), while the interface segment of conduit/tube in unit [32] may be fabricated from silicone rubber or a similar material.

As shown in detail in the figures, mechanical pump-tube interface unit [32] may couple to a pump [21] so as to bring the interface segment of conduit/tube into engagement with the peristaltic mechanism of the pump. According to some embodiments, a mechanical pump-tube interface unit [32] may be supplied as a pre-assembled, disposable kit, with or without tube segments [26] and [28]. Unit [32] may be constructed so as to enable an operator [34] to connect the unit to pump [21] stably and reliably by fitting the unit against the pump and snapping it into place with only light pressure. A connection between unit [32] and pump [21] may be self-aligning, such that operators are able to perform this operation with a single hand, after only minimal training. After use, unit [32] may be disengaged from pump [21] and may be discarded together with the tube.

According to some embodiments, a mechanical pump-tube interface unit may include mechanical elements/adaptations to connect and secure the interface unit to a pump. For example, a mechanical pump-tube interface unit may include latches and connectors matching hinges, latches and connectors upon a pump. Such mechanical elements and adaptations may be configured to connect the interface unit to the pump such that when the interface is attached to the matching pump the pumping mechanism of the pump is positioned to act upon the fluid in the tube causing it to flow through conduit/tube [as shown in FIG. 9, for example].

For example, a mechanical pump-tube interface unit [such as 32] may comprise a body [38], which may secure an interface segment of conduit/tube [10]. In the exemplary embodiment shown in the figures, interface segment of conduit/tube [10] may be connected to supply line [26] and patient line [28] by connectors [such as 48 and 50], respectively. Body [38] may have an elongated shape, corresponding to the linear configuration of the pumping mechanism [36]. As further described below, an interface segment of conduit/tube [10] may be secured within the mechanical pump-tube interface unit such that longitudinal and/or rotational movement of the interface segment of conduit/tube [10] may be prevented and/or inhibited. Mechanism [36] may comprise multiple fingers [56], which may move up and down to compress and release the interface segment of conduit/tube [10] in a predetermined cyclic pattern, so as to propel fluid downstream from supply line [26] to patient line [28]. Details of the operation of this sort of multi-finger peristaltic mechanism are described in the above-mentioned U.S. patent application Ser. No. 11/791,599 and in PCT Patent Applications PCT/IL2007/001398 and PCT/IL2007/001400, filed Nov. 13, 2007, whose disclosures are incorporated herein by reference.

Exemplary mechanical pump-tube interface unit [32] may comprise a hinge tongue [40] at one end of body [38] (in this case, the downstream end) and a catch receptacle [44] at the other (upstream) end. To attach unit [32] onto pump [21], the operator may first bring the hinge tongue [40] into engagement with a hinge type receptacle [42] on a body of the pump. As can be seen in the figure, in this position, unit [32] may be aligned on a plane of peristaltic mechanism [36], however, able to rotate within the plane about an axis defined by the hinge type receptacle. The operator may rotate the unit [32] about this axis, while the hinge tongue is engaged to the hinge type receptacle, until the catch on the pump body [46] engages and locks onto a catch receptacle [44] located on the mechanical pump-tube interface unit. The catch may be spring-loaded (or otherwise elastic) so that it slides over and then locks onto the catch receptacle as the operator presses unit [32] down against pump [21]. Once engaged and locked in this manner, movement of unit [32] may be substantially restricted in all directions. Unit [32] may subsequently be released from pump [21] by opening the catch, rotating the unit away from the pump and releasing the hinge tongue from the hinge type receptacle.

The rotational mode of assembly described above is advantageous in that it ensures accurate alignment of interface segment of conduit/tube [10] with mechanism [36], even in one-handed operation. Furthermore, as the position of interface segment of conduit/tube [10] within the unit [32] is secured (as further described below), the same locations on interface segment of conduit/tube [10] will contact the same locations of the pumping mechanism, thereby improving flow accuracy without the need for very careful insertion of the tube into the pump. The inventors have found that the combination of this sort of mechanical pump-tube interface unit with the type of peristaltic pump described in the above-mentioned patent applications gives ±2.5% accuracy or better in flow control.

The position of a hinge type receptacle [42] may be pre-adjusted so that the mechanical pump-tube interface unit [32] and interface segment of conduit/tube [10], when engaged and locked onto pump [21], may be properly located relative to fingers [56]. For example, the hinge type receptacle may be connected to pump body by a single screw, which may permit the receptacle to be moved and then tightened in place in a factory calibration procedure. This sort of calibration may be used to find the optimal balance between pressure buildup and energy consumption for propelling fluid at high pressure.

Furthermore, the above described architecture gives the operator a mechanical advantage (lever) in closing the catch receptacle against the catch, so that a relatively small force is needed to perform the connection. In a clinical version of system [20], the inventors have found that typically 1.7-2.4 kg of force is sufficient for this purpose.

Another advantage of a mechanical pump-tube interface unit [such as 32] and the mating structure on pump [21] is that they may ensure that the tube will be assembled onto the pump in the proper direction. As one type of mating connector is used at the upstream end of unit [32], and a different type of mating connector is used at the downstream end, correct alignment during attachment may become inherent and the risk of the operator accidentally attaching the unit in the reverse direction greatly reduced.

In an exemplary embodiment illustrated in the figures, the hinge type receptacle [42] has the form of a split axle, while the hinge tongue [40] has the form of a split saddle. At the other end of unit [32], catch receptacle [44] has the form of a split tooth, while catch [46] comprises a dual, concave catch. Interface segment of conduit/tube [10] thus may pass through the opening between the sides of tongue [40], receptacle [42], catch receptacle [44] and catch [46]. This particular configuration of the hinge and catch parts of pump [21] and unit [32] have been found to provide mechanical stability, durability and ease of assembly.

On the other hand, other configurations of the hinge and catch parts are also possible, as will be apparent to those skilled in the art, and are considered to be within the scope of the present invention. For example, the “male” and “female” elements on the mechanical pump-tube interface unit and pump body may be reversed, so that the hinge and catch components on the interface unit have the form of an axle and elastic catch, while the hinge and catch receptacles on the pump have the form of a saddle and tooth. Other suitable hinge and catch arrangements are described in the above-mentioned U.S. patent application Ser. No. 11/791,599.

In some cases, the peristaltic mechanism may include multiple fingers, which are driven to compress and release the conduit/tube in a predetermined cyclic pattern.

In some embodiments, a mechanical pump-tube interface unit may be shaped to match a shape of the pump mechanism it is intended to interface to. For example, when the peristaltic mechanism has a linear configuration, the mechanical interface may have an elongated shape corresponding to the linear configuration of the peristaltic mechanism.

In some embodiments, the pump body may include a rim [49] surrounding the peristaltic mechanism, and an interface segment of conduit (defined below) may include flanges or may include connectors having flanges [22], fixed to opposing ends of an interface segment of conduit/tube [10] or part of connectors affixed to the ends of an interface segment of conduit/tube, which flanges may lodge against the rim when the unit is attached to the pump. The infusion pump may further include a door or cover [55], which may close over the rim so as to enclose the peristaltic mechanism. Such a rim may have openings [41] shaped to receive a conduit/tube so that a connected conduit/tube extends through the openings when the door is closed. According to some embodiments, the flanges may be configured to lodge inside or outside the rim and have respective diameters that are larger than the openings so as to prevent/inhibit longitudinal and/or rotational motion of the conduit/tube after the door has been closed. According to further embodiments, one or both of the flanges may be secured/trapped by a trap of the mechanical pump-tube interface unit to prevent/inhibit longitudinal and/or rotational motion of the conduit/tube, as further described below.

Returning to the previous example, after assembly of mechanical pump-tube interface unit [32] onto pump [21], a cover [55] may be closed against a rim [49] over the unit for added security. A locking mechanism [55] on the cover may prevent accidental opening of the cover. Pump [21] may comprise a sensor for detecting whether cover [55] is closed, such as a magnetic sensor, which may detect the proximity of a magnet [57] attached to the cover. Until the operator is ready to close the cover, however, spring-loaded hinges [59] may hold the cover open so that it does not interfere with handling of the mechanical pump-tube interface unit.

According to some embodiments, a mechanical pump-tube interface unit may include an anti-free-flow mechanism [33], which may be configured to prevent flow of the fluid through the portion of the tube secured within the interface unit. The anti-free-flow mechanism may be adapted to automatically engage any time the interface unit is disconnected from a pump and automatically disengage whenever the unit is connected to a pump. According to some embodiments, the anti-free-flow mechanism may have an override mechanism to allow disengagement of the anti-free-flow mechanism without connecting the interface to the pump, i.e. while the interface unit is not connected. According to further embodiments, the mechanical pump-tube interface unit may further include an anti-free-flow override locking mechanism designed to lock the override mechanism of the anti-free-flow mechanism in an overriding position. According to yet further embodiments, such a lock may automatically unlock when the mechanical pump-tube interface unit is connected to a pump. Accordingly, a user may actuate and lock the override mechanism while the interface unit is disengaged from a pump. In this position the fluid may be free to flow through the conduit/tube without any further action from the user, however, according to these embodiments, if the mechanical pump-tube interface unit is connected to a pump, the override mechanism may automatically unlock such that if the interface unit is again disconnected from the pump, the anti-free-flow mechanism will engage normally and block fluid flow in the conduit/tube unless the override mechanism is actuated by the user once again.

Returning again to the previous example, mechanical pump-tube interface unit [32] may also comprise an anti-free-flow mechanism [33], which may, when engaged, block an interface segment of conduit/tube [10] residing within the interface unit, in order to prevent uncontrolled flow of infusion fluid into the patient's body. Mechanism [33] may be adapted to automatically engage any time the interface unit is disconnected from a pump and automatically disengage whenever the unit is connected to a pump. For example, springs [78] may automatically engage mechanism [33] when the interface unit is disconnected from the pump and a key on the pump body may disengage mechanism [33] when the interface unit is connected to the pump. Mechanical pump-tube interface unit [32] may further include an anti-free-flow override mechanism adapted to allow manual disengagement of anti-free-flow mechanism [33], while the interface unit is disconnected from the pump, allowing liquid flow through the conduit/tube without connection to the pump (e.g. by gravity). Mechanical pump-tube interface unit [32] may yet further include an anti-free-flow override locking mechanism designed to lock the override mechanism of the anti-free-flow mechanism in an overriding position. This lock may automatically unlock when the mechanical pump-tube interface unit is connected to a pump, so as to ensure that the mechanism engages (and prevents inadvertent free flow) when the interface unit is again disconnected from the pump. Details of this sort of anti-free-flow mechanism mechanism are described in the above-mentioned U.S. patent application Ser. No. 11/791,599 and in PCT Patent Application PCT/IL2007/001405, filed Nov. 13, 2007, both of which are incorporated herein by reference in their entirety.

According to some embodiments, there may be provided an interface segment of conduit/tube [10] designed to reside within a mechanical pump-tube interface unit. According to further embodiments, an interface segment of a conduit/tube may be comprised of a material suitable for interacting with a pump, such as silicon materials. It should be understood that the rest of the conduit/tube (i.e. a portion intended to reside outside the pump-tube interface unit) may be comprised of a different material (e.g. pvc).

According to some embodiments, an interface segment of conduit/tube may include or be connected, at one or both of its ends, to connectors adapted to connect the interface segment of conduit/tube to a supply line on one side and/or a patient line on the other. Such connectors [48 & 50] may include flanges [22] which may function as described above in relation to flanges, i.e. may lodge inside or outside a rim of the pump and have respective diameters that are larger than the openings so as to prevent longitudinal motion of the conduit/tube. It should be understood that other combinations of conduit sections are possible and equally relevant to the present description. For example, an interface segment of conduit/tube and a patient or supply line may be provided as one conduit/tube with a single connector at its end, i.e. an interface segment of conduit/tube and a supply or patient line may be supplied as a single tube to be inserted into and secured within the interface unit.

According to some embodiments, a mechanical pump-tube interface unit may include mechanical adaptations (traps) [37] adapted to secure/trap an interface segment of conduit/tube within the interface unit and prevent longitudinal and/or rotational movement, in relation to the interface unit, of an interface segment of conduit/tube residing within the interface unit. Such mechanical adaptations (traps) may be designed to act on the tube itself and/or upon one or both of the connectors at its ends.

For example [see FIGS. 4A-4B], a mechanical pump-tube interface unit may include u shaped grooves [37A] on one or both sides designed to receive the flanges on the end connectors and secure/trap them in place, thus preventing longitudinal movement of the interface segment of conduit/tube within the unit and possibly further preventing or inhibiting rotational movement of the interface segment of conduit/tube. According to further embodiments, a trap of the mechanical pump-tube interface unit may further comprise mechanical adaptations adapted to secure a connector within the trap. For example, an elastic or spring/tension based protrusion [39] may reside within the above described u shaped grooves. Thus, when a connector/flange is inserted into the groove, the protrusion may be forced away when the connector is inserted into the groove [as shown in FIGS. 6G-6H] and return once the connector/flange has passed it, so as to prevent the connector/flange from exiting the trap. Accordingly, the connector may “click”/“snap” into the trap. According to some embodiments, securing/trapping the interface segment of conduit/tube within the mechanical pump-tube interface unit, as described herein, may be performed by mechanical means, without any need to adhere the conduit/tube to the interface unit, e.g. by use of adhesives or welding processes.

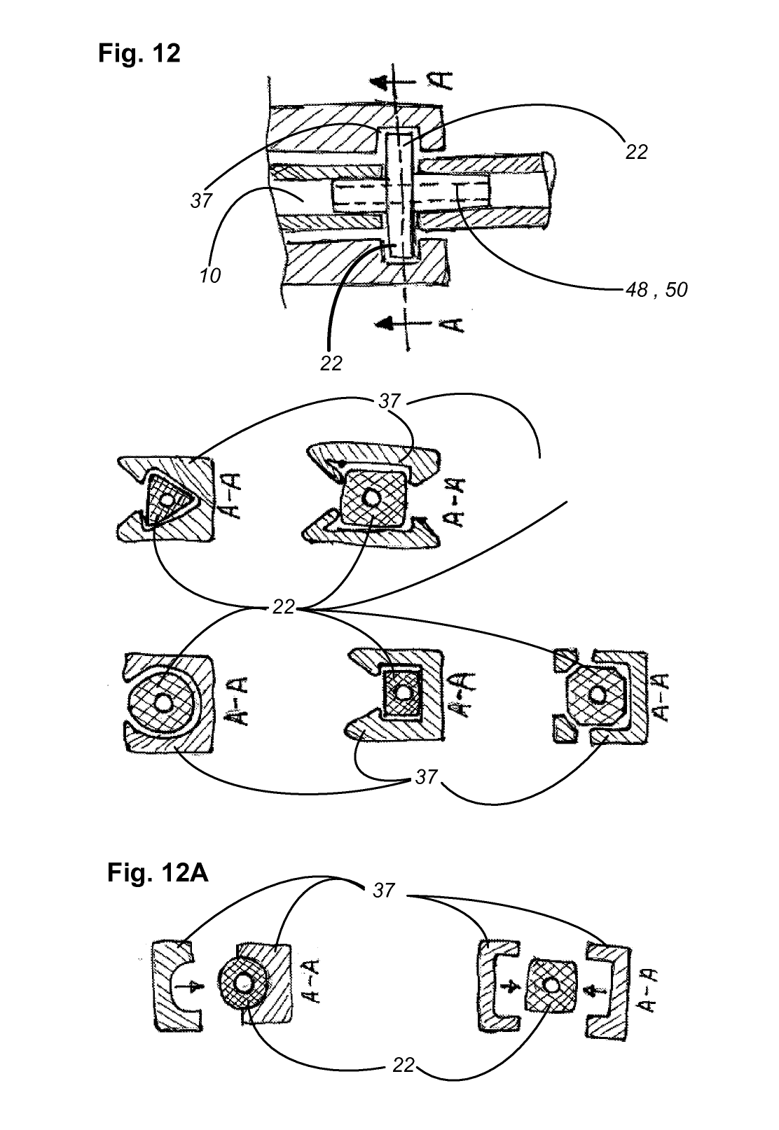

In another example, opposing sides of the mechanical pump-tube interface unit parts, on one or both ends, may each include a portion of a trap [see FIGS. 12-12A], e.g. a half circle groove, each matching a half shape of an end connector/flange. Accordingly, a mechanical pump-tube interface unit may be assembled with an interface segment of conduit/tube, attached to end connectors, already residing within the unit, wherein the end connector(s) are placed in between the half circle grooves, such that the grooves close upon the connector/flange and thus secure/trap the conduit/tube within the unit.

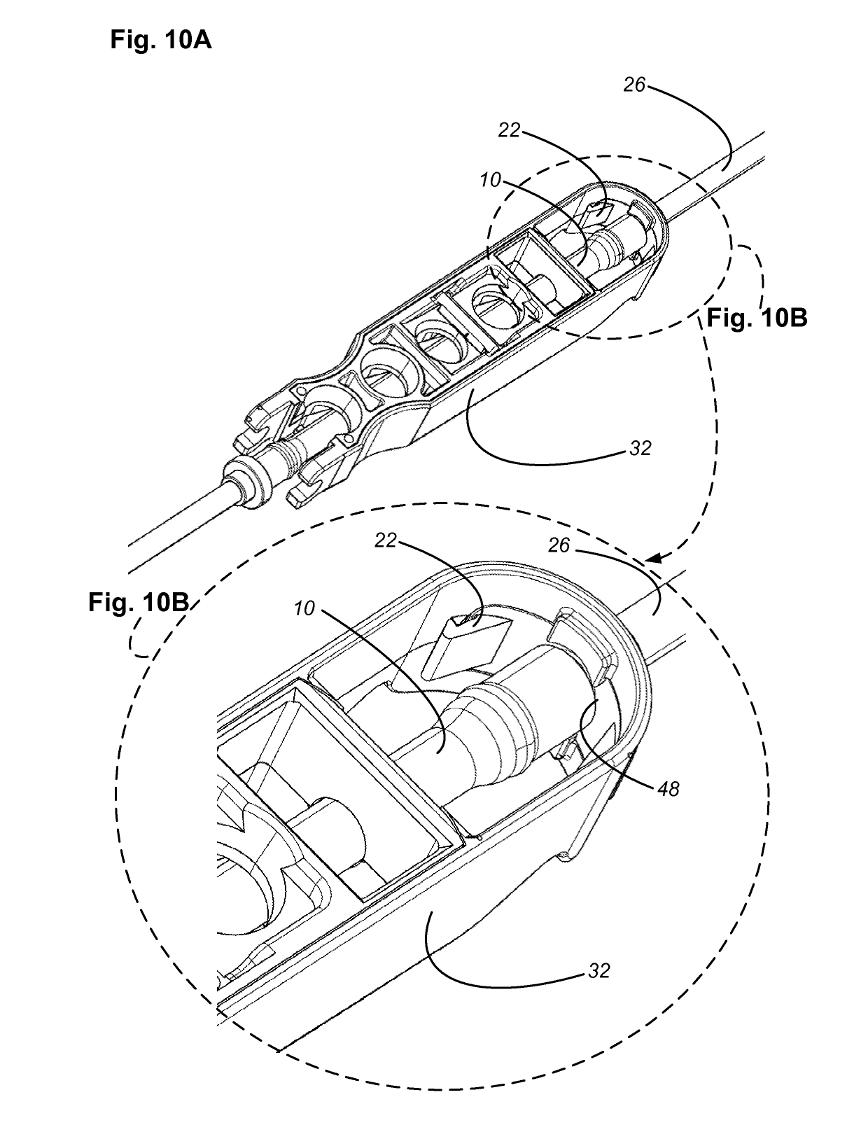

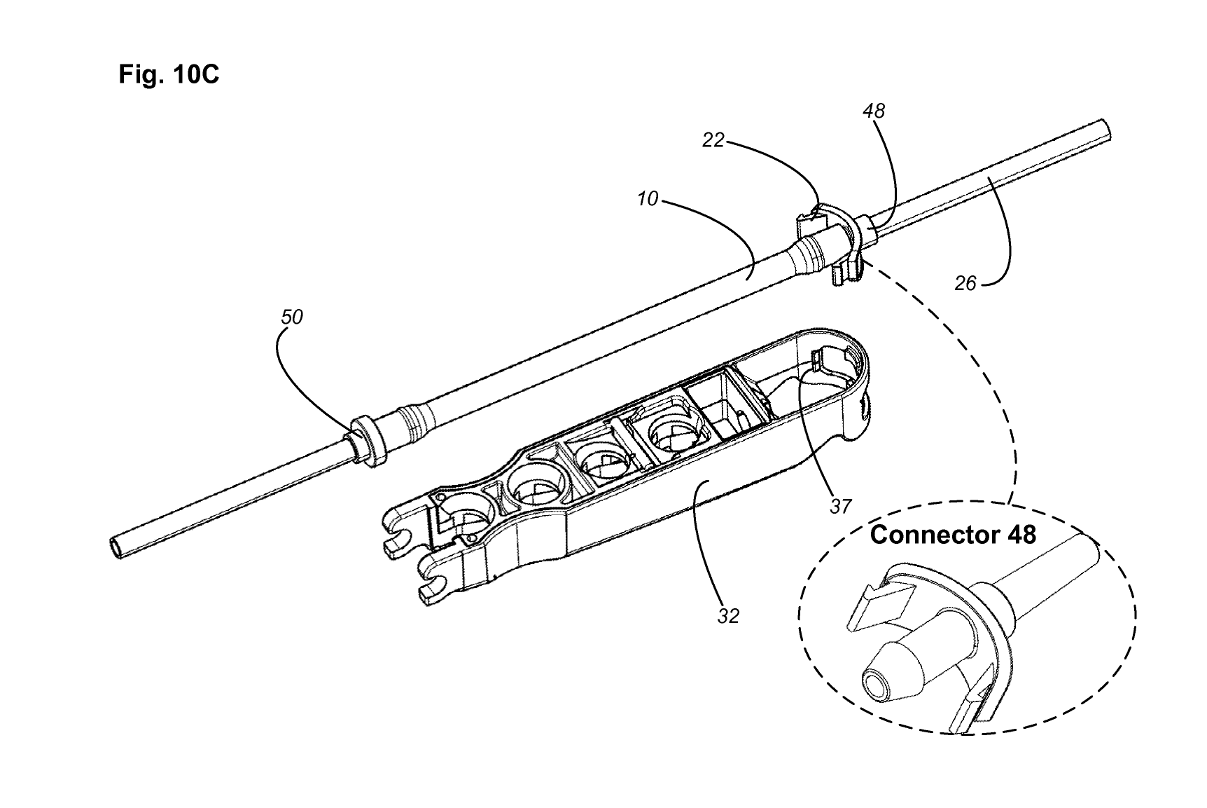

In another example, a front loading trap may be provided [see FIGS. 10A-10C & 11A-11H]. In such embodiments, a trap may include a circular opening through which an interface segment of tube may be inserted until the connector reaches the trap. The circular opening may be too small to allow a flange of a connector attached to the interface segment of conduit/tube to pass. A further mechanical element (trap—[37]) may prevent/inhibit the interface segment of conduit/tube from being retracted in the direction it was inserted. For example, a spring based rim or hook(s) [37] on the exterior side of the trap may catch the flange once it passes it/them and thereby prevent it from retreating the way it entered.

According to further embodiments, a flange of a connector used to secure/trap an interface segment of conduit/tube within a mechanical pump-tube interface unit may have a non-circular shape, e.g. a square shape, a rectangular shape, a hexagonal shape, and so on [see FIGS. 10(A-C) & 12]. Accordingly, the trap in which the flange is to be inserted may also have a matching, non-circular shape [see FIGS. 10(A-C) & 12]. In this manner rotational movement of a secured/trapped conduit/tube may be further inhibited/prevented. Clearly, a square flange within a square trap will be inhibited from rotating. According to some embodiments, other modifications of the flange and/or trap may be implemented to further prevent/inhibit rotational movement of a secured/trapped conduit/tube. For example, protrusions and/or a rough or ribbed surface within the trap (e.g. along the inside of a u-shaped groove of a trap) may inhibit rotational movement of a connector secured/trapped within. Equally, such protrusions, and/or rough or ribbed surfaces may be implemented upon the exterior surface of the flange to inhibit/prevent rotational movement, or both elements may include such adaptations.

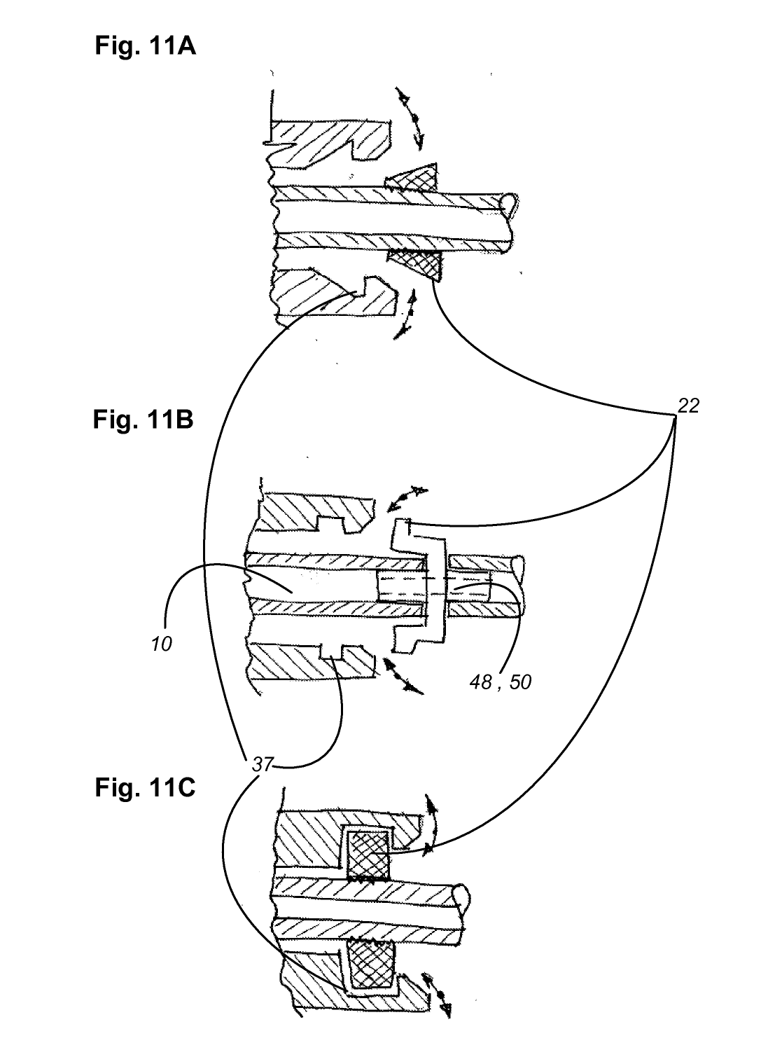

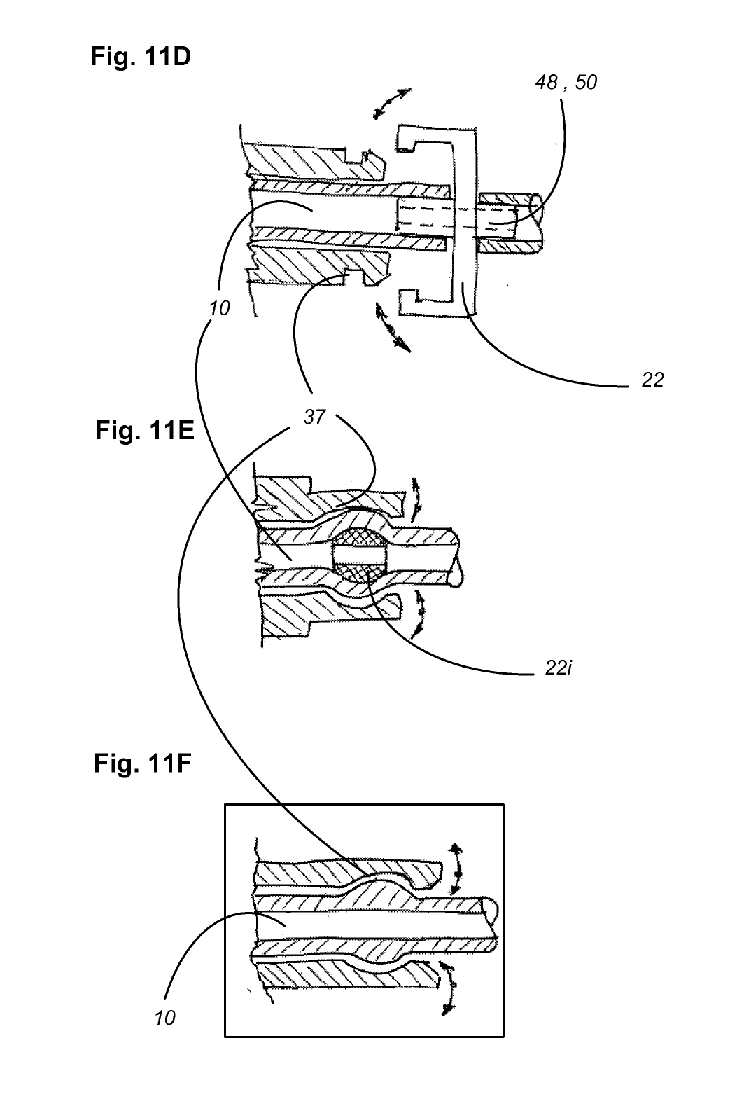

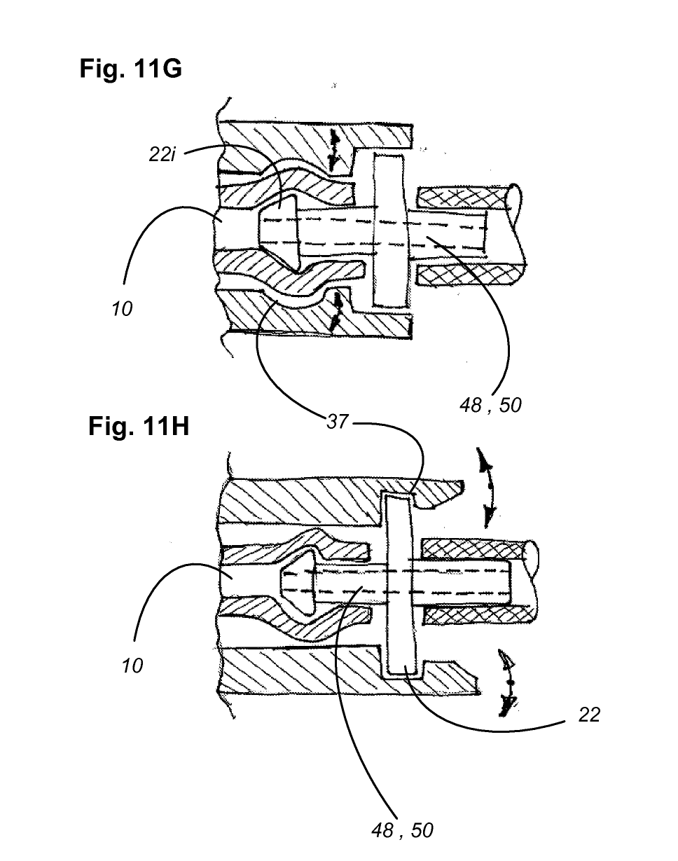

According to some embodiments, a connector may include a flange(s) [22i] designed to reside within an interface segment of conduit/tube the connector is connected to, thereby causing a protrusion in the conduit/tube [see FIGS. 11E-11G]. In such embodiments, a trap may act upon the protrusion in the conduit/tube instead of directly on the flange, as shown in FIGS. 11E-11G.

According to some embodiments, in addition or instead of a trap designed to act upon a connector, a mechanical pump-tube interface unit may include mechanical components adapted to act directly upon an associated interface segment of conduit/tube and secure/trap the interface segment of conduit/tube within the interface unit and prevent longitudinal and/or rotational movement, in relation to the interface unit, of the interface segment of conduit/tube residing within the interface unit. For example, a set of one or more protrusions into the channel in which the interface segment of conduit/tube may be included in a mechanical pump-tube interface unit. These protrusions may apply force on the tube preventing/inhibiting its movement once inserted into the interface unit, e.g. a set of protrusions opposing each other may “pinch” a conduit within the channel, thereby inhibiting its movement. According to further embodiments, such protrusions may be biased by tension into the channel, thereby improving their function.

Securing/Trapping the interface segment of conduit/tube within the mechanical pump-tube interface unit may prevent stretching of the conduit/tube which may be caused by users of the device during its use. Such stretching may result in changes and instability in the mechanical characteristics of the conduit/tube, which in turn may cause deviations in flow and inaccuracy of pressure sensor measurements. Clearly, pressure measurements and flow regulation performed by an associated pump are based on calculations which factor the mechanical properties of the conduit/tube on which the pumping mechanism is acting. Accordingly, if the conduit/tube is stretched (e.g. as a result of misplacement) and thus its mechanical properties altered or rendered instable, these calculations will be accordingly altered and rendered inaccurate. Equally, as flow regulation is based on such calculations, any deviation/instability in the mechanical properties of the conduit/tube may lead to deviations, inaccuracy and/or instability in the flow regulation. In light of these considerations, securing/trapping the interface segment of conduit/tube as described herein, promotes accuracy and stability of the pumping process by preventing the possibility of misplacing and thus stretching the conduit/tube.

A further advantage of securing an interface segment of conduit/tube within a mechanical pump-tube interface unit is that the same points on the conduit/tube will contact the pumping and sensing elements of the pump throughout disconnection and reconnection of the unit to the pump, i.e. throughout the life of the mechanical pump-tube interface unit. As the pumping action of the pumping mechanism against the conduit/tube affects and modifies over time the mechanical properties of those points on the tube being pressed, these modifications must be factored when performing the above mentioned calculations in relation to pressure measurements and flow regulation. By ensuring that the same points on the conduit/tube will contact the pumping and sensing elements of the pump throughout disconnection and reconnection of the unit to the pump, i.e. throughout the life of the interface unit, accurate and continuous profiles of the mechanical properties of these points on a given conduit/tube may be maintained. Thereby, accurate and stable pressure sensing and flow regulation may be achieved as long as the same points on the conduit/tube are positioned opposite the pumping and pressure sensing elements of the pump throughout. If, however, the conduit/tube were to move (e.g. rotate or shift longitudinally) during disconnection and reconnection, different points of the tube, having different mechanical properties would subsequently contact the pumping and sensing elements of the pump, thereby causing inaccuracy and instability in pressure sensing and flow regulation. In light of these considerations, securing/trapping the interface segment of conduit/tube as described herein, promotes accuracy and stability of the pumping process by preventing movement of the conduit/tube when disconnected and reconnected to the pump, thereby maintaining the same contact points between the pumping and sensing mechanisms of the pump and the conduit/tube.

According to further embodiments, a method of assembling a mechanical pump-tube interface unit may be provided. According to some embodiments, an interface segment of conduit/tube, including connectors, may be attached to (i.e. assembled within) a mechanical pump-tube interface unit, sterilized and packaged together. The interface segment of conduit/tube may be secured within the interface unit in its final position by appropriate mechanical fasteners, grooves and/or any other suitable mechanical configuration. Subsequently, when ready to be used, the mechanical pump-tube interface unit may first be attached to a supply and patient line by means of the connectors on the ends of the interface segment of conduit/tube and then connected to a pump as described above. The interface segment of conduit/tube may thus become an integral part of the interface unit. Securing an interface segment of conduit/tube within a mechanical pump-tube interface during assembly of the interface unit may prevent movement and/or misalignment of the interface segment of conduit/tube, in relation to the interface unit, during shipping, storage, unpackaging and assembly to the other components of the infusion system (presumably occurring at the user's site).

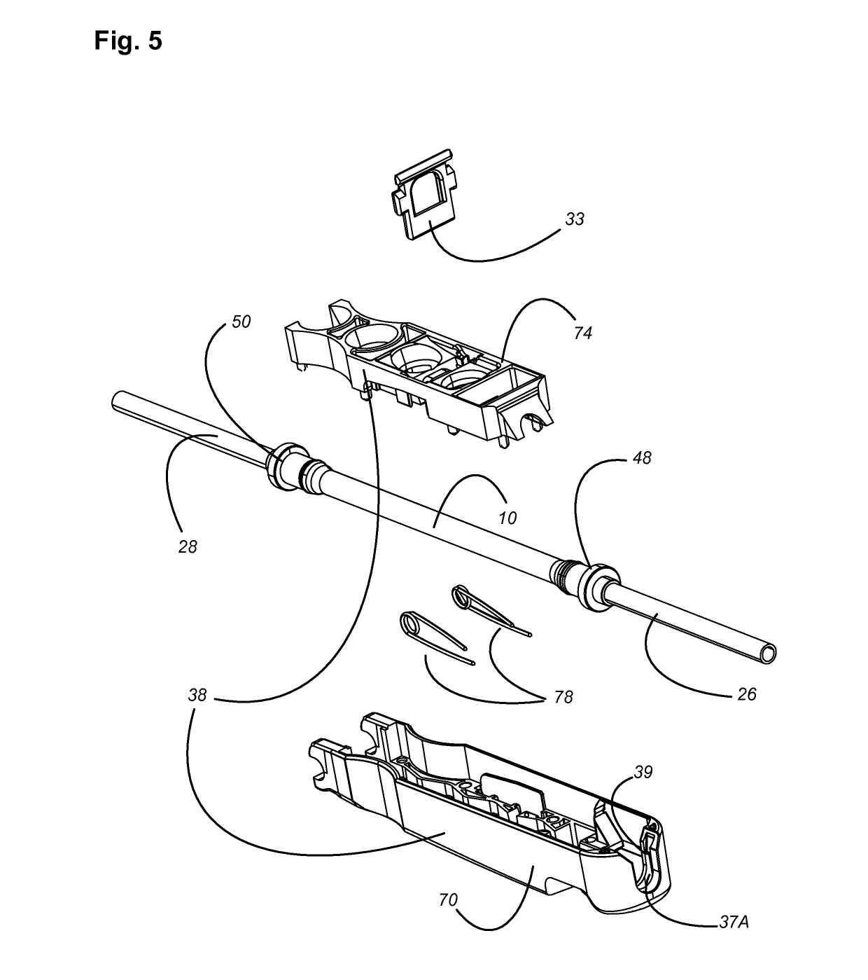

FIG. 5 includes illustrations of exemplary components of an exemplary mechanical pump-tube interface unit [32], in accordance with some embodiments of the present invention. Mechanical pump-tube interface Unit [32] comprises an outer shell [70] and an inner shell [74], which define a central channel for receiving an interface segment of conduit/tube [10]. Holes [58] which are provided to allow a pump mechanism to interact with the interface segment of conduit/tube within the central channel can be seen on the inner shell. Trap [37A] in the form of a u shaped groove can be seen on the end of the outer shell.

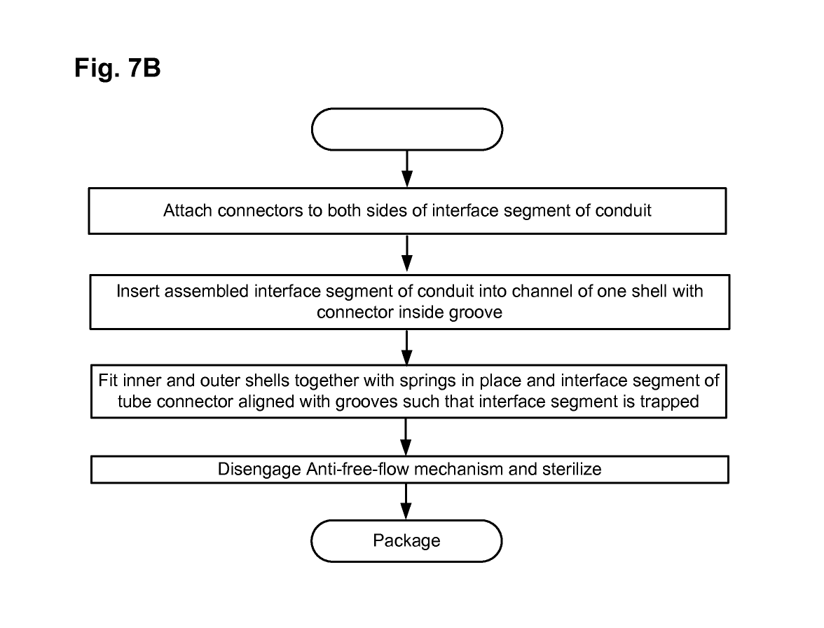

Turning now to FIGS. 6A-6K and 7, to assemble unit [32], shells, inner [70] and outer [74], are first fitted together and Anti-free-flow mechanism [33] may be mounted in a slot in unit [32] against springs [78], which hold the mechanism in its closed position. Subsequently or in parallel, a connector [48] is attached to one end of an interface segment of conduit/tube [10] [see FIGS. 6A-6B]. The interface segment of conduit/tube [10] is then inserted into the channel [FIG. 6D] and the connector [48] on the supply line side is inserted into trap [37A] and clicked/snapped in place [FIGS. 6E-6H], such that the interface segment of conduit/tube [10] is now secured/trapped in place (note: the anti-free-flow mechanism may be locked in a disengaged position to allow insertion of the conduit). After insertion of the interface segment of conduit/tube into the mechanical pump-tube interface unit, connector [50] may be connected to the open (unsecured) end of the interface segment of conduit/tube [FIGS. 6I-6K]. For sterilization the override mechanism of the anti-free-flow mechanism may be engaged to allow flow of the sterilization agents through the conduit/tube. Alternatively, an interface segment of tube may first be inserted into the channel and both connectors connected afterwards. The processes illustrated in FIGS. 6A-6K may be performed by hand. It should be understood, however, that one or more or all of the steps illustrated therein and described herein may be performed by appropriate machinery.

Alternatively, in embodiments in which a trap is implemented by opposing grooves on either shell, to assemble unit [32], first connectors [48 & 50] may be attached to both ends of an interface segment of conduit/tube [10]. The interface segment of conduit/tube [10] may then be placed in the channel of one shell with the connector inserted into one groove, the shells [70 and 74] may then be fitted together with the connector on the end of the interface segment of conduit/tube [10] aligned with the grooves, thus trapping the tube portion securely in place. Finally, Anti-free-flow mechanism [33] is mounted in a slot in unit [32] against springs [78], which hold the mechanism in its closed position. In other embodiments, connectors at both sides of the interface segment of tube may be secured in traps of the mechanical pump-tube interface unit. The above described methods of assembly may then further include steps of trapping the second connector into its respective trap.

There is additionally provided, in accordance with some embodiments of the present invention, a method for infusion, including providing a mechanical pump-tube interface unit, which holds and secures/traps an interface segment of conduit/tube and includes a hinge tongue and a catch receptacle. The hinge tongue may be inserted into a hinge type receptacle, which defines a hinge axis, on an infusion pump. The mechanical pump-tube interface unit may be rotated about the hinge axis while the hinge tongue is engaged to the hinge type receptacle until the catch on the pump is secured upon the catch receptacle on the mechanical pump-tube interface unit, so as to bring the tube into engagement with a peristaltic mechanism of the infusion pump and secure it there. The infusion pump may be actuated while the tube is in engagement with the peristaltic mechanism so as to propel a fluid through the tube.

Although the embodiment shown in the figures uses a particular type of linear finger-based mechanism, the principles of the present invention may similarly be applied to peristaltic pumps using other types of mechanisms, including cam-based mechanisms, as well as circular mechanisms. It will thus be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

We claim:

1. A mechanical interface for interfacing between a pump and a fluid conduit, said interface comprising:
a housing for securing the fluid conduit, said housing being adapted to detachably connect to the pump, such that when said housing is connected to the pump a pumping mechanism of the pump is positioned to act upon fluid within the conduit secured within said housing;
a mechanical trap to receive a connector attached to the conduit, said trap being adapted to trap the connector within the trap thereby inhibiting movement, in relation to said housing, of the conduit connected to the connector.

2. The interface according to claim 1, wherein said trap inhibits longitudinal movement of the conduit, in relation to said housing, once the connector is trapped by said trap.

3. The interface according to claim 1, wherein said trap inhibits rotational movement of the conduit, in relation to said housing, once the connector is trapped by said trap.

4. The interface according to claim 1, wherein said trap is comprised of a u shaped groove into which said connector is inserted.

5. The interface according to claim 4, wherein said trap further comprises a protrusion within the u shape groove, which protrusion inhibits said connector from exiting said trap.

6. The interface according to claim 5 wherein said protrusion is biased by tension into the u shape.

7. The interface according to claim 3, wherein the connector has a shape matching a shape of the trap and said matching shapes facilitate the inhibition of rotational movement.

8. The interface according to claim 1, wherein the connector snaps into said trap.

9. The interface according to claim 1, wherein said mechanical trap is comprised of opposing grooves which are assembled on opposing sides of the connector, thereby trapping the connector between them.

10. The interface according to claim 1, wherein said trap secures said conduit within said housing, such that identical points upon said conduit contact the pumping mechanism of the pump when said interface is disconnected and reconnected to the pump.

11. The interface according to claim 1, wherein said trap prevents stretching of the conduit by a user when connecting said interface to the pump.

12. A method for assembling an interface between a pump and an infusion kit, said method comprising:
assembling a housing for securing a fluid conduit, said housing being adapted to detachably connect to the pump, wherein the housing is designed such that when connected to the pump a pumping mechanism of the pump is positioned to act upon fluid within a conduit secured within said housing;
attaching connectors to one or both sides of an interface segment of conduit, said connectors being adapted to connect to an infusion tube;
inserting the interface segment of conduit into the assembled housing;
inserting at least one of the connectors into a trap of the housing, the trap being adapted to trap the connector within the trap thereby inhibiting movement of the interface segment of conduit, in relation to the housing.

13. The method according to claim 12, wherein said trap inhibits longitudinal movement of the conduit, in relation to said housing, once the connector is trapped by said trap.

14. The method according to claim 12, wherein said trap inhibits rotational movement of the conduit, in relation to said housing, once the connector is trapped by said trap.

15. The method according to claim 12, wherein said trap is comprised of a u shaped groove into which said connector is inserted.

16. The method according to claim 15, wherein said trap further comprises a protrusion within the u shape groove, which protrusion inhibits said connector from exiting said trap.

17. The method according to claim 16, wherein said protrusion is biased by tension into the u shape.

18. The method according to claim 14, wherein the connector has a shape matching a shape of the trap and said matching shapes facilitate the inhibition of rotational movement.

19. The method according to claim 12, wherein inserting a connector includes snapping the connector into said trap.

20. A system for medical infusion, comprising:
a pump having an exterior pumping mechanism;
an interface segment of conduit having a connector on one end, the connector being adapted to connect the conduit to an infusion tube;
a housing adapted to secure the interface segment of conduit, said housing being further adapted to detachably connect to said pump, such that when said housing is connected to said pump the pumping mechanism is positioned to act upon fluid within the conduit secured within said housing;
a mechanical trap within said housing to receive the connector, said trap being adapted to trap the connector within the trap thereby inhibiting movement of the conduit, in relation to said housing.
